(19) United States
(12) Patent Application Publication
(10) Pub. No.: US 2025/0381413 A1
(43) Pub. Date: Dec. 18, 2025

(54) FEEDTHROUGH SUPPORT FEATURES

(71) Applicant: Cardiac Pacemakers, Inc., St. Paul, MN (US)

(72) Inventors: James Michael English, Cahir (IE); Moira B. Sweeney, St. Paul, MN (US); Robert Allen Jones, Lake Elmo, MN (US); Liam Ryan, Co. Waterford (IE); Kamran Equbal, Clonmel (IE)

(21) Appl. No.: 19/232,591

(22) Filed: Jun. 9, 2025

Related U.S. Application Data
(60) Provisional application No. 63/659,598, filed on Jun. 13, 2024.

(51) Int. Cl. A61N 1/375; A61N 1/39; B33Y 80/00; H01R 13/03; H01R 27/02; H01R 43/20

(52) U.S. Cl. CPC A61N 1/3754; A61N 1/39622; B33Y 80/00; H01R 13/03; H01R 27/02; H01R 43/20; H01R 2201/12

(57) ABSTRACT

An implantable medical device can include a housing including electronic devices within the housing, a header attached to the housing and including one or more bores, and a plurality of connectors extending from the housing to the header and coupled to a plurality of electrical contacts within the one or more bores, wherein at least one of the plurality of connectors includes a first end coupled to a feedthrough pin on the housing and second end coupled to one of the plurality of electrical contacts, wherein the first end includes a contoured shape configured to provide a feedthrough connection section having a non-uniform cross-section.

### CLAIM OF PRIORITY

This application claims the benefit of U.S. Provisional Application No. 63/659,598, filed on Jun. 13, 2024, which is hereby incorporated by reference in its entirety.

### TECHNICAL FIELD

Various embodiments described herein relate to apparatus, systems, and methods associated with implantable medical devices.

### BACKGROUND

An ambulatory medical device, such as an implantable medical device (IMD), can be configured for implant in a subject, such as a patient. An IMD can be configured to be coupled to a patient's heart such as via one or more implantable leads. Such an IMD can obtain diagnostic information or generate therapy to be provided to the patient, such as via the coupled implantable lead.

In one configuration, IMDs have a header that is coupled to a container that houses much of the electronics of the IMD. The header can be used to couple a conductor of the implantable lead with circuitry within the implantable device. In some examples, one or more electrical connectors can extend from feedthroughs of the housing to the header. Such connectors requires consistent placement for proper performance of the device and a robust connection of the connectors to the feedthrough pins and the header contacts.

### SUMMARY

Example 1 can include subject matter such as an implantable medical device including a housing including electronic devices within the housing, a header attached to the housing and including one or more bores, and a plurality of connectors extending from the housing to the header and coupled to a plurality of electrical contacts within the one or more bores, wherein at least one of the plurality of connectors includes a first end coupled to a feedthrough pin on the housing and second end coupled to one of the plurality of electrical contacts, wherein the first end includes a contoured shape configured to provide a feedthrough connection section having a non-uniform cross-section.

In Example 2, the subject matter of Example 1 can optionally include wherein the contoured shape includes a conical shape having a first wide end on a connection side of the connection section and extending to an open hole on an outer surface of the connection section such that the feedthrough pin is exposed through the open hole.

In Example 3, the subject matter of any one or more of Examples 1-2 can optionally include wherein contoured shape includes a thinned portion of the feedthrough connection section that is thinner in a cross-section than a main portion of the connector, wherein the thinned portion includes a flattened area of the connection section.

In Example 4, the subject matter of any one or more of Examples 1-3 can optionally include wherein the contoured shape includes a pocket pad defining a generally circular recessed portion on a feedthrough pin connection side of the connector to receive the feedthrough pin and leading to a thinned portion located on an outer side of the connector.

In Example 5, the subject matter of any one or more of Examples 1˜4 can optionally include wherein the contoured shape includes a pocket pad defining a generally lengthened oval shaped recessed portion on a feedthrough pin connection side of the connector to receive the feedthrough pin and leading to a thinned portion located on an outer side of the connector.

In Example 6, the subject matter of any one or more of Examples 1-5 can optionally include wherein the contoured shape includes a thinned portion of the feedthrough connection section that is thinner in a cross-section than a main portion of the connector, wherein the thinned portion includes flat pads.

In Example 7, the subject matter of any one or more of Examples 1-6 can optionally include wherein the thinned portion includes flat pads that are wider that a main portion of the connector.

In Example 8, the subject matter of any one or more of Examples 1-7 can optionally include wherein the contoured shape includes a thinned portion of the feedthrough connection section that is thinner in a cross-section than a main portion of the connector, wherein the contoured shape of the thinned portion includes swaged contact pads.

In Example 9, the subject matter of any one or more of Examples 1-8 can optionally include wherein the contoured shape includes a thinned portion of the feedthrough connection section that is thinner in a cross-section than a main portion of the connector, wherein the feedthrough connection section includes an inverse pocket structure including the thinned portion with gussets located around a periphery of the thinned portion to stiffen the feedthrough connection section.

In Example 10, the subject matter of any one or more of Examples 1-9 can optionally include wherein the contoured shape includes a hole extending though the feedthrough connection section.

In Example 11, the subject matter of any one or more of Examples 1-10 can optionally include wherein the hole includes a chamfered portion around a periphery of the hole on an outer surface of the feedthrough connection section.

Example 12 can include an implantable medical device including a housing including electronic devices within the housing, a header attached to the housing and including one or more bores, and a plurality of connectors extending from the housing to the header and coupled to a plurality of electrical contacts within the bore, wherein at least one of the plurality of connectors includes a first end coupled to a feedthrough pin on the housing and second end coupled to one of the plurality of electrical contacts, wherein the second end includes a contoured shape configured to provide an electrical contact connection section having a non-uniform cross-section.

In Example 13, the subject matter of Example 12 can optionally include wherein the contoured shape includes a thinned portion that is thinner than a main portion of the connector, wherein the thinned portion includes a flat surface having a width that is wider than the main portion of the connector.

In Example 14, the subject matter of any one or more of Examples 12-13 can optionally include wherein the contoured shape includes an opening including a slot in the connector to allow for a line-of-site weld connection.

In Example 15, the subject matter of any one or more of Examples 12-14 can optionally include wherein the contoured shape includes a thinned portion that is thinner than a main portion of the connector, wherein the thinned portion includes a chamfered shape narrowing down to a thin connection section on an edge of the connector.

Example 16 can include subject matter such as a method for positioning a connector assembly relative to an implantable medical device including positioning a connector assembly to connect between a plurality of feedthrough pins of a housing and a header of the implantable medical device, the connector assembly comprising a plurality of connectors extending from a common sacrificial section, the connectors being pre-formed into a 3-dimensional shape such that one or more of the connectors lie in a different plane from a side view than others of the plurality of connectors; coupling a first end of each of the connectors to a corresponding one of the plurality of feedthrough pins; coupling a second end of each of the connectors to a corresponding electrical contact in the header; and removing the sacrificial section.

In Example 17, the subject matter of Example 16 can optionally include wherein the connector assembly includes a main connector portion which is separated from the sacrificial section by a break-off feature.

In Example 18, the subject matter of any one or more of Examples 16-17 can optionally include wherein the connector assembly includes a standoff extending from a back surface of the connector proximate the break-off feature.

In Example 19, the subject matter of any one or more of Examples 16-18 can optionally include wherein sacrificial portion includes one or more keyed holes to couple to a fixture configured for aligning the connector assembly with the feedthrough pins and the electrical contacts.

In Example 20, the subject matter of any one or more of Examples 16-19 can optionally include forming the connector assembly using additive manufacturing.

In Example 21, subject matter (e.g., a system or apparatus) may optionally combine any portion or combination of any portion of any one or more of Examples 1-20 to comprise “means for” performing any portion of any one or more of the functions or methods of Examples 1-20, or at least one “non-transitory machine-readable medium” including instructions that, when performed by a machine, cause the machine to perform any portion of any one or more of the functions or methods of Examples 1-20.

This summary is intended to provide an overview of subject matter of the present patent application. It is not intended to provide an exclusive or exhaustive explanation of the disclosure. The detailed description is included to provide further information about the present patent application. Other aspects of the disclosure will be apparent to persons skilled in the art upon reading and understanding the following detailed description and viewing the drawings that form a part thereof, each of which are not to be taken in a limiting sense.

### BRIEF DESCRIPTION OF THE DRAWINGS

FIG. 1 shows an example implantable medical device, in accordance with one embodiment.

FIG. 2 shows a detail of a header portion of the implantable medical device, in accordance with one embodiment.

FIG. 3 shows a side view of the header portion of the implantable medical device, in accordance with one embodiment.

FIG. 4 shows a top view of a connector assembly, in accordance with one embodiment.

FIG. 5 shows a side view of the connector assembly, in accordance with one embodiment.

FIG. 6 shows a perspective view of a connection side of a feedthrough connection section, in accordance with one embodiment.

FIG. 7 shows a perspective view of a front side of the feedthrough connection section, in accordance with one embodiment.

FIG. 8 shows a view of a front side of a feedthrough connection section, in accordance with one embodiment.

FIG. 9 shows a perspective view of a front side of a feedthrough connection section, in accordance with one embodiment.

FIG. 10 shows a perspective view of a front side of a feedthrough connection section, in accordance with one embodiment.

FIG. 11 shows a perspective view of a connection side of a feedthrough connection section, in accordance with one embodiment.

FIG. 12 shows a perspective view of a connection side of a feedthrough connection section, in accordance with one embodiment.

FIG. 13 shows a perspective view of a front side of a feedthrough connection section, in accordance with one embodiment.

FIG. 14 shows a perspective view of a front side of a feedthrough connection section, in accordance with one embodiment.

FIG. 15 shows a perspective view of a front side of a feedthrough connection section, in accordance with one embodiment.

FIG. 16 shows a perspective view of a front side of a feedthrough connection section, in accordance with one embodiment.

FIG. 17 shows a perspective view of a front side of a feedthrough connection section, in accordance with one embodiment.

FIG. 18 shows a perspective view of an electrical contact connection section, in accordance with one embodiment.

FIG. 19 shows a perspective view of an electrical contact connection section, in accordance with one embodiment.

FIG. 20 shows a perspective view of an electrical contact connection section, in accordance with one embodiment.

FIG. 21 shows a perspective view of an electrical contact connection section, in accordance with one embodiment.

FIG. 22 shows a perspective view of an electrical contact connection section, in accordance with one embodiment.

FIG. 23 shows a perspective view of an electrical contact connection section, in accordance with one embodiment.

FIG. 24 shows a perspective view of an electrical contact connection section, in accordance with one embodiment.

FIG. 25 shows a perspective view of a connection side of a portion of a connector assembly, in accordance with one embodiment.

FIG. 26 shows a series of side view of a portion of a plurality of connector assemblies, in accordance with various embodiments.

FIG. 27 shows a perspective view of a front side of a lower portion of a connector assembly, in accordance with one embodiment.

FIG. 28 shows a perspective view of a lower portion of a connector assembly, in accordance with one embodiment.

FIG. 29 shows a perspective view of a connector assembly within a fixture, in accordance with one embodiment.

FIG. 30 shows a method of assembly of a connector assembly to portions of an implantable medical device, in accordance with one embodiment.

### DETAILED DESCRIPTION

In the following detailed description, reference is made to the accompanying drawings that form a part hereof and in which are shown, by way of illustration, specific embodiments in which the invention may be practiced. These embodiments are described in sufficient detail to enable those skilled in the art to practice the invention. Other embodiments may be utilized and structural, logical, and electrical changes may be made.

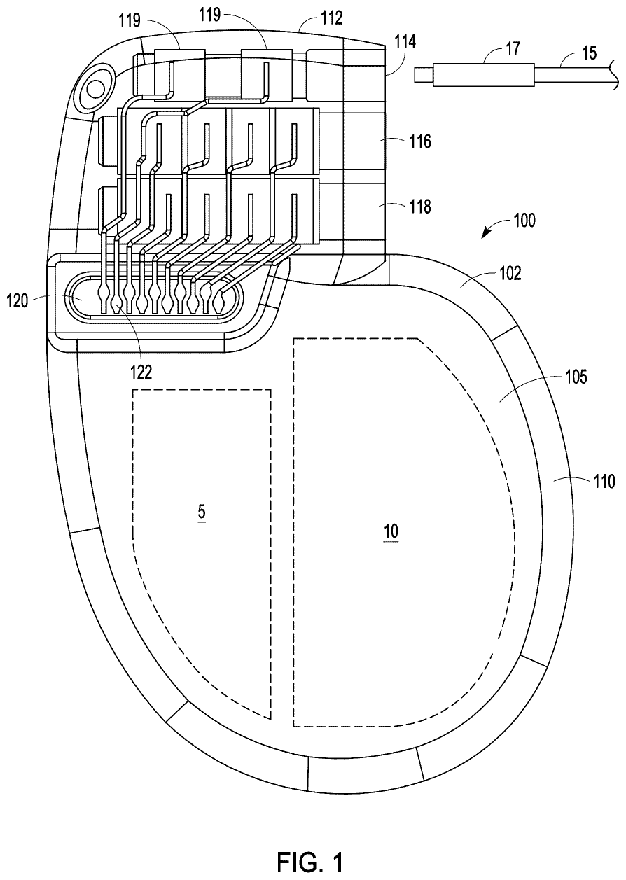

FIG. 1 shows an implantable system 100 including an implantable medical device 102, in accordance with one embodiment. The implantable medical device 102 includes a pulse generator 105 and at least one implantable lead 15. The pulse generator 105 includes a housing 110 and a header 112 mounted to the housing 110. The pulse generator 105 can be implanted into a subcutaneous pocket made in the wall of a patient's chest. Alternatively, the pulse generator 105 can be placed in a subcutaneous pocket made in the abdomen, or in other locations. Pulse generator 105 can include electronic devices such as a power supply 5 including a battery, a capacitor, and other components housed in the housing 110. The pulse generator 105 can further include other electronic devices 10 such as microprocessors to provide processing, evaluation, and to deliver electrical shocks and pulses of different energy levels and timing for defibrillation, cardioversion, and pacing to a heart in response to cardiac arrhythmia including fibrillation, tachycardia, heart failure, and bradycardia.

The header 112 can include one or more bores 114, 116, 118 to receive implantable leads, such as the implantable lead 15. The implantable lead 15 can include electrodes on a distal end to provide therapy to a body and include a terminal pin 17 on the proximal end to couple to the bore 114, 116, 118. At least one electrical conductor is disposed within the lead 15 and extends from the proximal end to the electrode. The electrical conductor carries electrical currents and signals between the pulse generator 105 and the distal electrode.

Contacts on the terminal pin 17 can contact electrical contacts 119, such as connector blocks, within the bores 114, 116, 118 to allow signals and therapy to be delivered to and from the electrodes in a body to the electronics 5, 10 within the housing 110. The electrical contacts 119 can be connected by electrical connectors 122 to a feedthrough assembly 120 to electrically communicate between the lead 15 and the electronics within the housing 110.

In one example, the header 112 can be formed from a polymer material. A polymer can provide a number of desirable features, such as biocompatibility, strength, resilience, and ease of manufacturing. In one example, the header 112 is molded separately from the housing 110, and later bonded to the housing 110 using an adhesive. In a second example, the header 112 can be molded in place (overmolded) and contacts a surface of the housing 110 during a curing or hardening process.

In other embodiments, the implantable system 100 can also be suitable for use with implantable electrical stimulators, such as, but not limited to, neuro-stimulators, skeletal stimulators, central nervous system stimulators, or stimulators for the treatment of pain.

In this example, the plurality of connectors 122 extend from the feedthrough assembly 120 and are coupled to different electrical contacts 119 within the bore. As noted above, during manufacture it is important to control the positioning of the connectors 122 within the header. The connectors requires consistent placement for proper performance of the device 102. Moreover, a robust connection between the connectors 122 and the feedthrough pins and the electrical contacts 119 is desirable.

Some present implantable devices utilize separately placed and positioned connector wires for each connector. Such a plurality of separate wires is difficult to manage and correctly place during manufacturing. Some devices utilize a stamped 2-dimensional connector assembly which require the user to bend and manipulate the connector wires during manufacture to correctly position the wires.

The present system can utilize additive manufacturing to form a connector assembly. As will be detailed below, the present system allows for varied shapes of the connector assembly as a whole to provide for improved manufacture. Moreover, connection pads on each end of the connectors can be designed to provide a more robust weld connection between the connectors and the feedthrough pins and the connector blocks of the header.

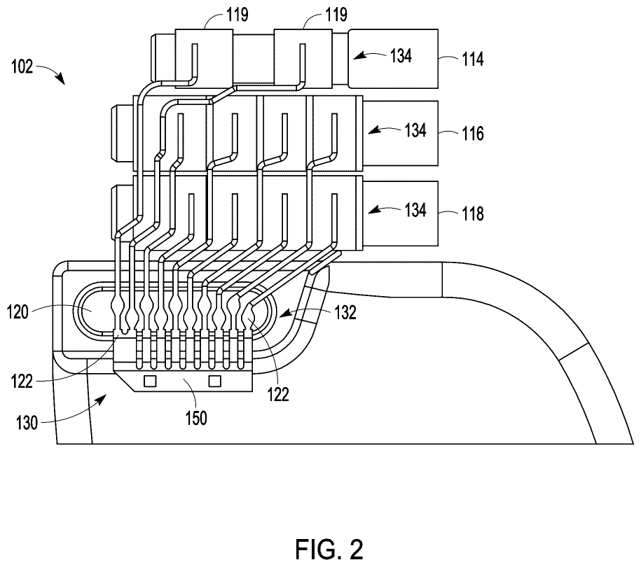

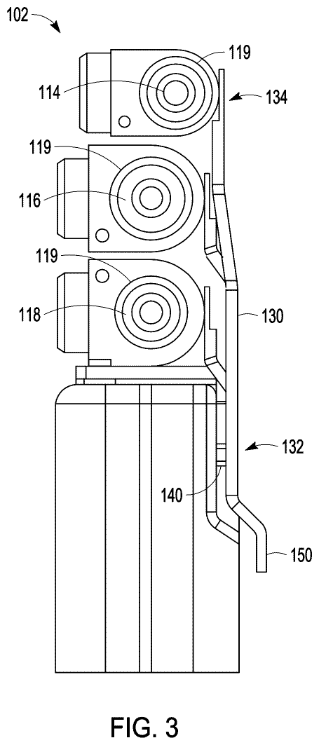

FIG. 2 shows a detail of the header portion of the implantable medical device 102 during manufacture, in accordance with one embodiment; and FIG. 3 shows a side view of the header portion of the implantable medical device 102.

In this example, a connector assembly 130 can include an additive manufactured connector assembly comprising the plurality of connectors 122 extending from a shared, common sacrificial section 150. Here, each of the plurality of connectors 122 includes a first end having a feedthrough connection section 132 coupled to a feedthrough pin 140 of the feedthrough assembly 120 on the housing 110 and a second end including an electrical contact connection section 134 coupled to one of the plurality of electrical contacts 119 located in the bores 114, 116, 118. The connector assembly 130 is performed such that each connector 122 is precisely positioned to connect to a given feedthrough pin and a given electrical contact 119.

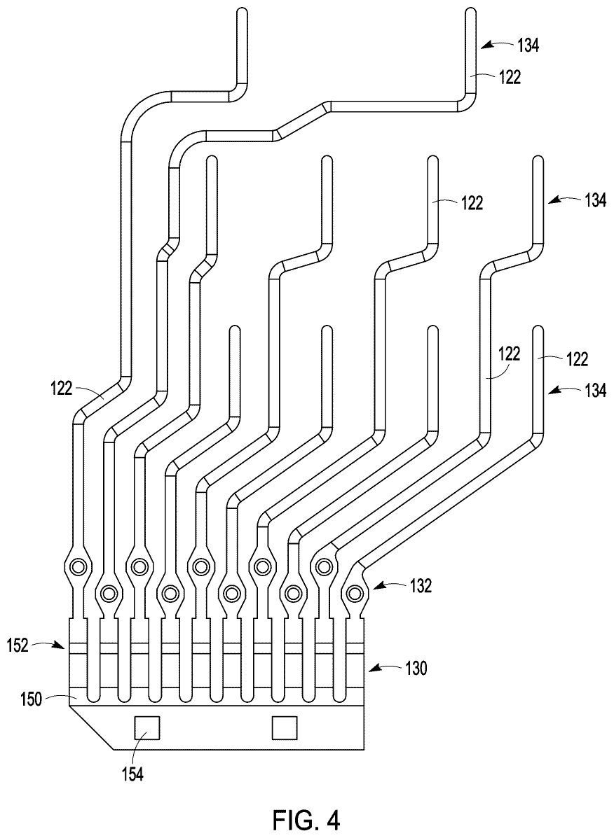

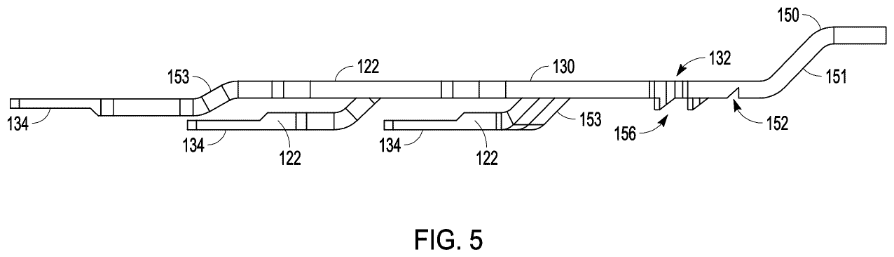

Referring now also to FIG. 4 which shows a top view of the connector assembly 130, and FIG. 5 which shows a side view of the connector assembly 130, further details of the present connector assembly 130 will be discussed.

As noted above, the connector assembly 130 can include an additive manufacturing formed connector assembly comprising the plurality of connectors 122 extending from the common sacrificial section 150. The connectors 122 can be pre-formed into a 3-dimensional shape during the additive manufacturing process such that one or more of the connectors 122 lie in a different plane from a side view than others of the plurality of connectors 122.

Thus, as shown in FIG. 5, the sacrificial section 150 can define an S-curve 151 bending down to down to a break-off feature 152. The S-curve 151 allows the connector assembly 130 to be properly positioned relative to the feedthrough pins and the electrical contacts 119 during manufacture while leaving room for grasping the sacrificial section 150 to break it off from the main body of the electrical connectors 122. (See FIG. 3). After the connectors 122 are coupled to the feedthrough pins and the electrical contacts 119, the sacrificial section 150 is removed from the connector assembly 130 by bending at the break-off feature 152. This results in the device as shown in FIG. 1.

As will be further detailed below, one or more standoffs 156 can be provided on the connection side of the connector assembly 130 to rest against the feedthrough assembly 120 so as to position the connectors 132 at a proper height relative to the feedthrough pins 140 and to provide leverage for bending the sacrificial section 150. Moreover, the sacrificial section 150 can include one or more keyed holes 154 to couple to a fixture configured for aligning the connector assembly 130 with the feedthrough pins 140 and the electrical contacts 119.

The various separate connectors 122 extending from the common sacrificial section 150 are formed, such as by additive manufacturing, to define various bends 153 to bring the second end electrical contact connection sections 134 of various connectors 122 into different planes from other connectors 122. This pre-forming of the connector assembly 130 allows for precise location of each electrical contact connection section 134 and allows for ease of manufacturing when connecting the second ends of the connector assembly 130 to the electrical contacts 119 in the header since the user does not have to manipulate each connector 122 to get the connector to a proper position. All the connectors 122 connected to the sacrificial section 150 are pre-formed in all 3 dimensions so as to be properly positioned in all 3 dimensions to easily attach to the electrical contacts 119.

As noted, the present design can include forming the connector assembly 130 using additive manufacturing. The connector assembly 130 can be formed of Niobium or other biocompatible conductor materials such as Ti, Pt, etc. In other uses for outside the body, the connector assembly 130 can be formed of any conductive material.

As noted, various shapes and contoured 3D designs can be utilized for the connection sections 132, 134 by using the additive manufacturing process that cannot be realized by stamping. The shapes of the connection sections can be non-uniform in at least one dimension. For example the connector can have a thinned portion, an opening, or be shaped in some fashion such that the overall shape is not uniform. Moreover, the present manufacturing technique allows for connections sections 132, 134 to have a variable, non-uniform cross sectional area. This is in contrast with present connectors formed by stamping where each connection section has a uniform depth, width, and height. The present connection sections can have a contoured, non-uniform shape and cross-section to optimize the connection to the feedthroughs or electrical contacts of the device.

Thus, the connection sections 132, 134 can be formed having a contoured shape configured to provide a connection section having a non-uniform cross-section relative to the main portion of the connector 122 across a length and/or width of the connector section 132, 134. For example, one or more of the connection sections 132, 134 can be formed to provide at least one of: 1) an opening, such as a hole, through the connection section to provide for a line-of-site weld and to allow for positional control of the connection section relative to its connection point, or 2) a thinned connection portion that is thinner in a cross-section than a main portion of the connector 122 to allow for a better weld while still allowing for the thicker main portion to have a low impedance connection. Examples of various contoured connection sections 132, 134 will be discussed below.

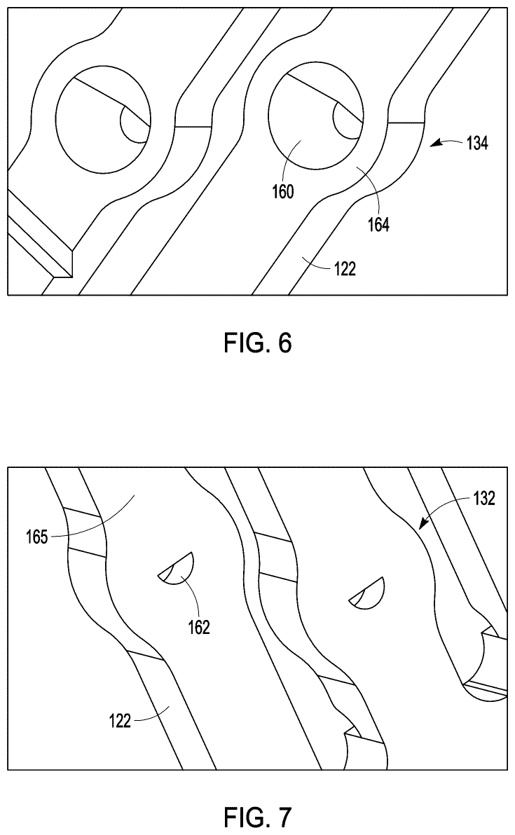

For example, FIG. 6 shows a perspective view of a connection side 164 of a feedthrough connection section 132, in accordance with one embodiment; and FIG. 7 shows a perspective view of the front side of the feedthrough connection section 132.

In this example, the feedthrough connection section 132 can include a contoured, 3-dimensional shape configured to provide an opening such as a hole 162 through connection section 132 to provide a line-of-site weld and to allow for positional control of the feedthrough connection section 132 of the connector 122 relative to the feedthrough pin 140. When a line-of-site connection is utilized, a zero-gap joint is provided where the connection section 132 and the feedthrough pin 140 can be melted together simultaneously without the heat having to pass through the top surface first.

In this example, the contoured shape at the first connector section 132 can include a conical shape 160 on a connection side 164 of the feedthrough connection section 132 defining a first wide open end on the connection side 164 and narrowing down to the open hole 162 on an outer surface 165 of the feedthrough connection section such that a feedthrough pin is exposed through the open hole 162. As noted, this contoured shape for the connector section 132 provides for a line-of-site weld scenario where the weld can be applied simultaneously to the feedthrough pin and the connector section 132 at the same time. This allows for a more robust connection. Moreover, the conical shape 160 of the connection section can be designed to draw the connector section 132 directly over the feedthrough pin if the connector section 132 is slightly off-center when first placed down.

Further in this example, the connection section 132 can be widened relative the main body of the connector 122. Moreover, the connection sections 132 can have a staggered structure such that every other connector section 132 is higher or lower than its adjacent neighbor connector section on the first end of the connector assembly 130.

FIG. 8 shows a view of a front side of a feedthrough connection section 132, in accordance with one embodiment.

In this example, the contoured shape of the connection section 132 at the first end includes a hole 166 extending through the connector section 132 from the back, connection side, through to the outer surface. The hole 166 can be formed in a widened portion 168 of the connection section 132 that is wider than a main body of connector 122. This contoured shape for the connection section 132 provides for a line-of-site weld scenario where the weld can be applied simultaneously to the feedthrough pin (which will extend through the hole 166) and the feedthrough connection section 132 at the same time.

Again, the connector sections 132 can have a staggered structure such that every other connection section 132 is higher or lower than its adjacent neighbor connector section on the first end of the connector assembly.

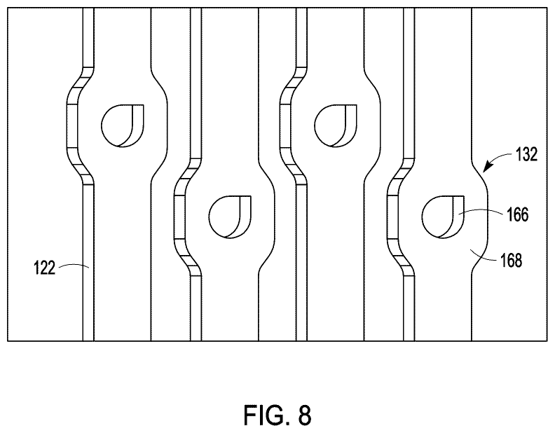

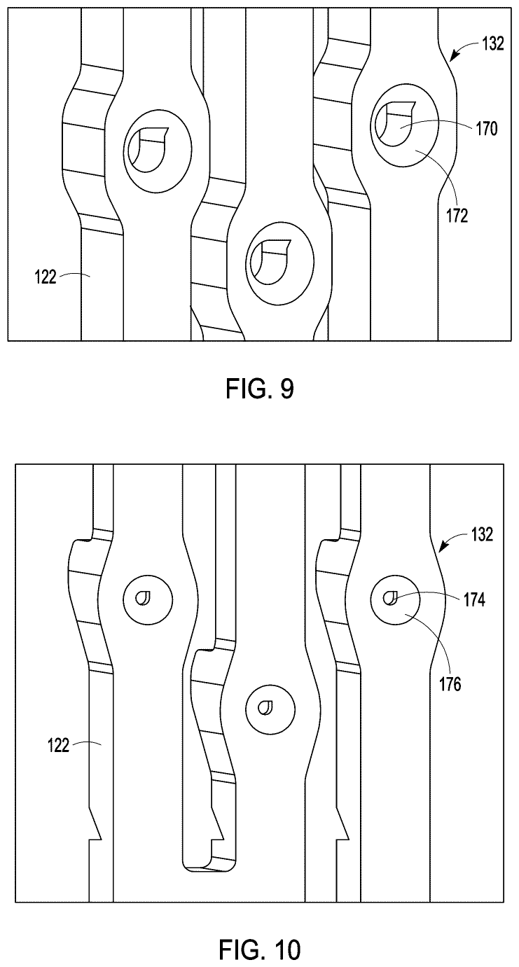

FIG. 9 shows a perspective view of a front side of a feedthrough connection section 132, in accordance with one embodiment.

This example is similar to the embodiment of FIG. 8 and certain details will be omitted. Here, the contoured shape of the connection section 132 includes a hole 170 having a chamfered surface 172 around a periphery of the hole 170. Again, the hole 170 provides for a line-of-site weld and positional control of the connection section 132 relative to the feedthrough pin which will extend through the hole 170. In some examples, the chamfer can provide for a better weld connection.

FIG. 10 shows a perspective view of a front side of a feedthrough connection section 132, in accordance with one embodiment. The embodiment of FIG. 10 is similar to IG. 9, but the hole 174 can be smaller than the hole 170 of FIG. 9. This connector also can include a chamfer 176 around a periphery of the hole 174.

As noted, some embodiments for the connection section 132 can be designed to provide a connection section 132 that has a thinner cross-section than a main portion of the connector 122. The main portion of the connector 122 is the section located between the feedthrough connection section 132 and the electrical contact connection section 134. Providing a thinner section at the connection section 132 provides for a robust weld joint between the feedthrough connection section 132 of the connector 122 and the feedthrough pin 140 since the weld energy only has to go through the thin portion before reaching the feedthrough pin. Specifically, the thinner portion allows for a better weld connection while the thicker main portion of the connector 122 allows for lower impedance.

For example, FIG. 11 shows a perspective view of the connection side 164 of a feedthrough connection section 132, in accordance with one embodiment.

In this example, the contoured shape of the connection section 132 includes a pocket pad 178 defining an open cut-out section on the back connection side 164 of the connector 122 leading to a thin-walled portion 180, that is not open on a front surface but instead acts as the connection point for the feedthrough pin to the connector 122. The thin-walled portion 180 defines a thin surface that is thinner than the main body of the connector 122 and thinner than the rest of the connection section 132. This design provides for a thinned connection section 132 that is thinner than a main body of the connector 132.

As noted, this thinned-walled connection surface 132 provides for a better weld at the thinned section 180 because the weld energy only has to go through the thin wall portion 180 before reaching the feedthrough pin, which will be abutting against the surface of the thin wall portion 180 when the connection section 132 is positioned over the feedthrough pin. Meanwhile, the relatively thicker main body of connector 122 provides for lower impedance. Thus, the thicker section of the main body of connector 122 has a lower impedance while the thinned portion 180 provides a section that is easier to weld to the feedthrough and makes for a better connection. Moreover, the pocket pad 178 can provide positional guidance for placing the connection section 132 over the feedthrough pin since the feedthrough pin will be received within the pocket pad 178 and abut against thin portion 180.

Accordingly, this embodiment defines a generally circular recessed portion defining the pocket pad 178 on a feedthrough pin connection side of the connector 122 to receive the feedthrough pin.

FIG. 12 shows a perspective view of a connection side of a feedthrough connection section 132, in accordance with one embodiment.

This example is similar to FIG. 11, but here pocket pads 179 and a thin-walled portion 182 define a generally lengthened oval shaped recessed portion on the feedthrough pin connection side 164 of the connector 122 to receive the feedthrough pin. By providing increased length of the pocket pad 179, this design allows for increased movement in the longitudinal axis of the connector 122 so as to provide for better tolerance control during assembly by allowing for tolerance errors in the positioning and placement of the pocket pad 179 over the feedthrough pin.

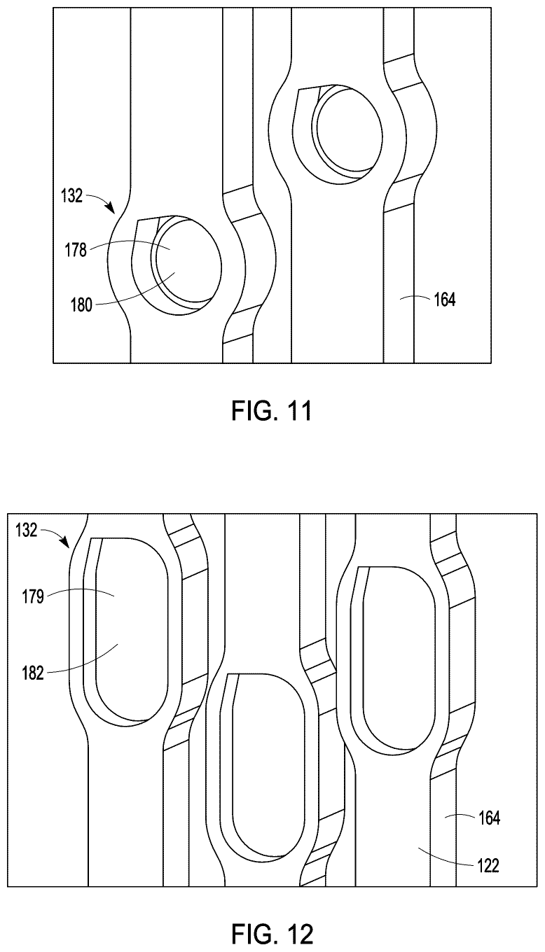

FIG. 13 shows a perspective view of a front side of a feedthrough connection section 132, in accordance with one embodiment.

In this embodiment, the contoured shape of the connection section 132 can include a straight connector that includes a thinned section 184 that is thinner at the connection section 132 than at a thicker portion 185 defining the main body portion of the connector 122. Thus, the thin section 184 is thinner than the main portion of the connector 122. As noted above, thinning the connection section 132 allows for more robust welding since it easier to connect to the feedthrough pin through thin material, while the thicker portion of the connector 122 allows for lower impedance.

In some embodiments, the thinned section 184 can be formed after the additive manufacturing is finished by using a post-processing technique such as stamp pressing. In other examples, the thinned section 184 can be formed during the additive manufacturing process.

FIG. 14 shows a perspective view of a front side of a feedthrough connection section 132, in accordance with one embodiment.

In this example the contoured shape of the connection section 132 can include a thin-walled portion 186, as above. However, in this example an outer gusset 188 around a periphery of the thin-walled portion 186 can be provided. The gusset 188 can include a cut out area within the body of the connection section 132 extending around the periphery of the thinned portion 186. This structure provides for a less fragile design since the gusset 188 stiffens the thinned connection section 132.

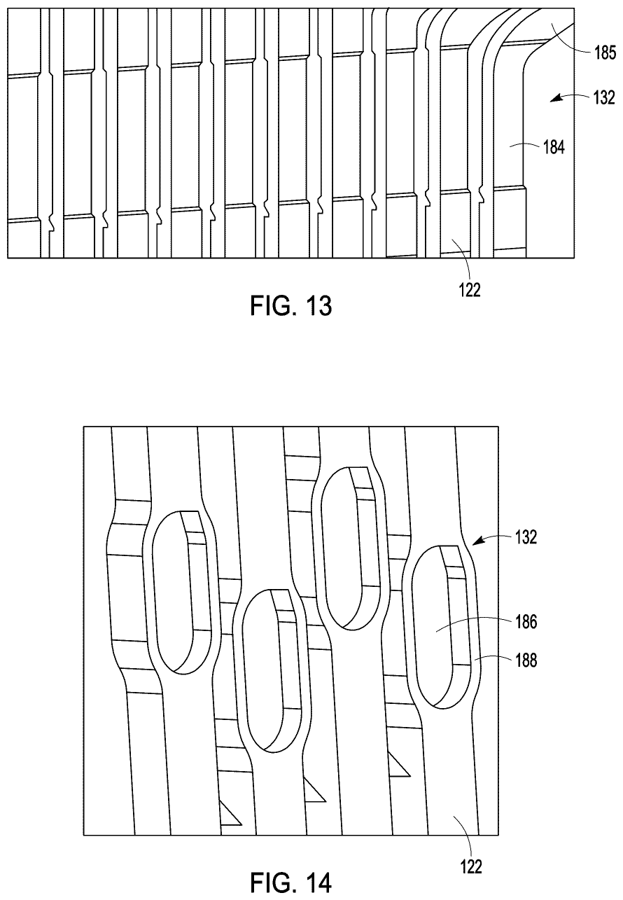

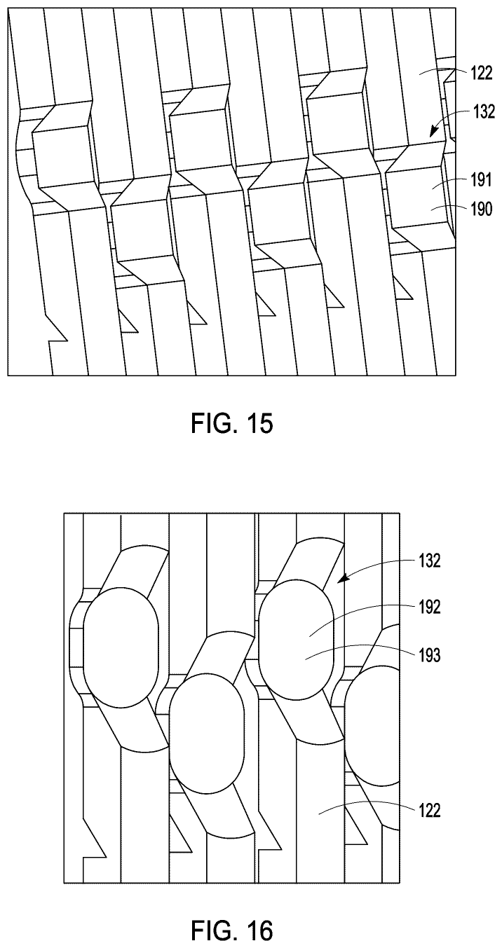

FIG. 15 shows a perspective view of a front side of a feedthrough connection section 132, in accordance with one embodiment.

In this example, the contoured shape of the connection section 132 can include a thinned section 190 defining flat pads 191 for connecting to the feedthrough pins, where the thinned section 190 has a thinner cross-section than a main portion of the connectors 122. In this example, the flat pads 191 are formed in a rectangular shape. In one example, the connection sections 132 of adjacent connector 122 can be positioned in an alternating staggered pattern.

FIG. 16 shows a perspective view of a front side of a feedthrough connection section 132, in accordance with one embodiment.

This example is similar to FIG. 15, including thinned section 192 defining flat pads 193. But in this example, the flat pads 193 can be formed as round or oval in shape. In one example, the flat pads 193 can be wider that a main portion of the connector 122.

FIG. 17 shows a perspective view of a front side of a feedthrough connection section 132, in accordance with one embodiment.

In this example, the contoured shape of the connection section 132 can include a thinned section 196 similar to the embodiment of FIG. 13. In this example the connection pads 194 are provided where the connection pads 194 are both thinned and are swaged so that the connection sections are wider than a main body of the connector 122. The swaging of the connection pad 194 allows the user to know where to apply the weld so as to connect to the feedthrough pin and also allows for more welding area to provide better tolerance of any mis-fits between the connection section 132 and the feedthrough pin.

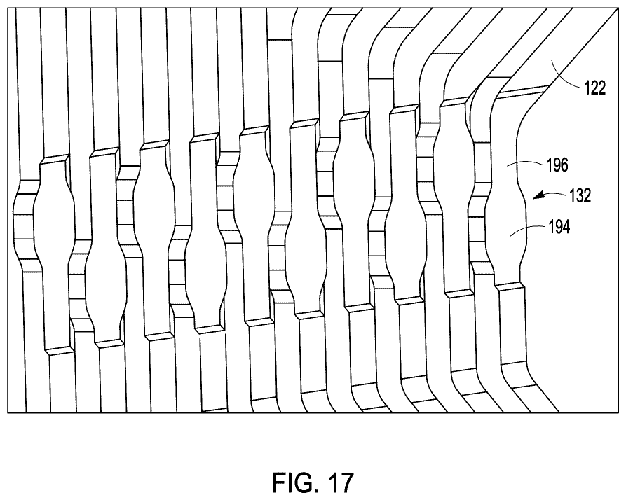

FIG. 18 shows a perspective view of an electrical contact connection section 134, in accordance with one embodiment.

Here, the electrical contact connection sections 134 for connecting to the electrical contacts 119 in the header (FIG. 2) can include a contoured 3-dimensional shape configured to provide a connection portion that includes a thinned portion defining a flat connection pad 202 that has a thinner cross-section than a main portion of the connector 122. The thinned portion defining the connection pad 202 allows for a more robust weld to the electrical contact 119 since the weld only needs to go through the thinned pad 202 before reaching the contact 119.

In this example, the width of the pad 202 can be about the same width as the main body of the connector 122.

FIG. 19 shows a perspective view of an electrical contact connection section 134, in accordance with one embodiment. This example is similar to the embodiment of FIG. 18, but here the thinned portion defines a wide connection pad 204 that is wider than the main portion of the connector 122. This wider area can allow for better tolerance of any mis-fits between the pad 204 and the contact 119.

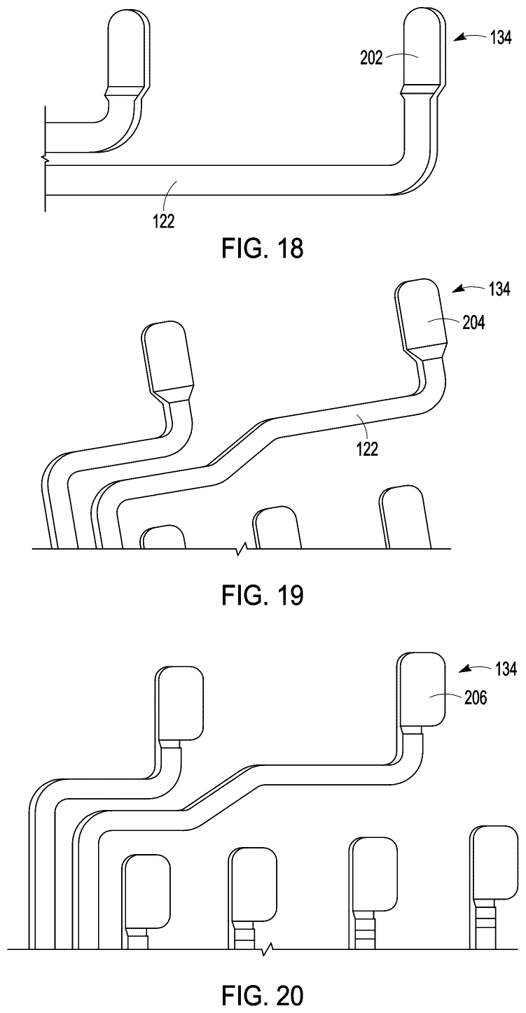

FIG. 20 shows a perspective view of an electrical contact connection section 134, in accordance with one embodiment.

This example is similar to the embodiment of FIG. 18, but here the thinned portion defines a flat pad 206 that is offset from a longitudinal axis of the main body of the connector 122.

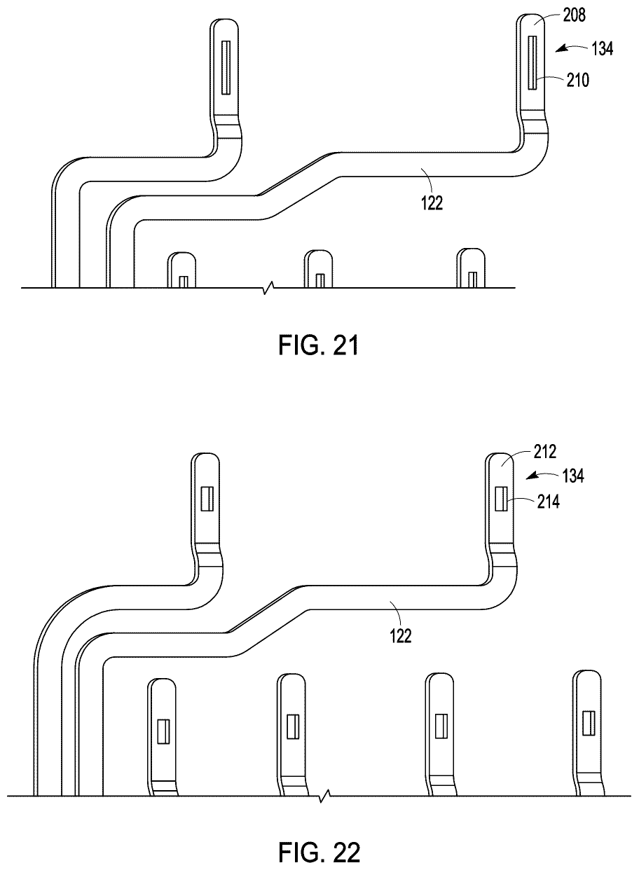

FIG. 21 shows a perspective view of an electrical contact connection section 134, in accordance with one embodiment.

In this embodiment, the electrical contact connection section 134 can include an opening through the electrical contact connection section 134 to provide for a robust weld joint between the connection section 134 of the connector 122 and the electrical contact by allowing for line-of-site weld connection between the two surfaces. As discussed above, the line-of-site weld allows for the two surface to be welded together simultaneously.

For example, in FIG. 21, a slot 210 can be provided in the second end at a connection section 208. The slot 210 extends all the way through the connection section 208 such that the electrical contact 119 is exposed through the slot 210 when the connection section 134 is in position over the contact 119 during manufacture.

FIG. 22 shows a perspective view of an electrical contact connection section, in accordance with one embodiment. This example is similar to FIG. 21, but a shorter slot 214 is shown in a connection section 212.

In either of these two options, other through-holes, such as round or oval though holes can also be provided. Again, the through-holes allow for a line-of-site weld to the electrical contact 119. Moreover, in these examples, the connections sections 208, 212 can be as thick as the main body of the connector 122.

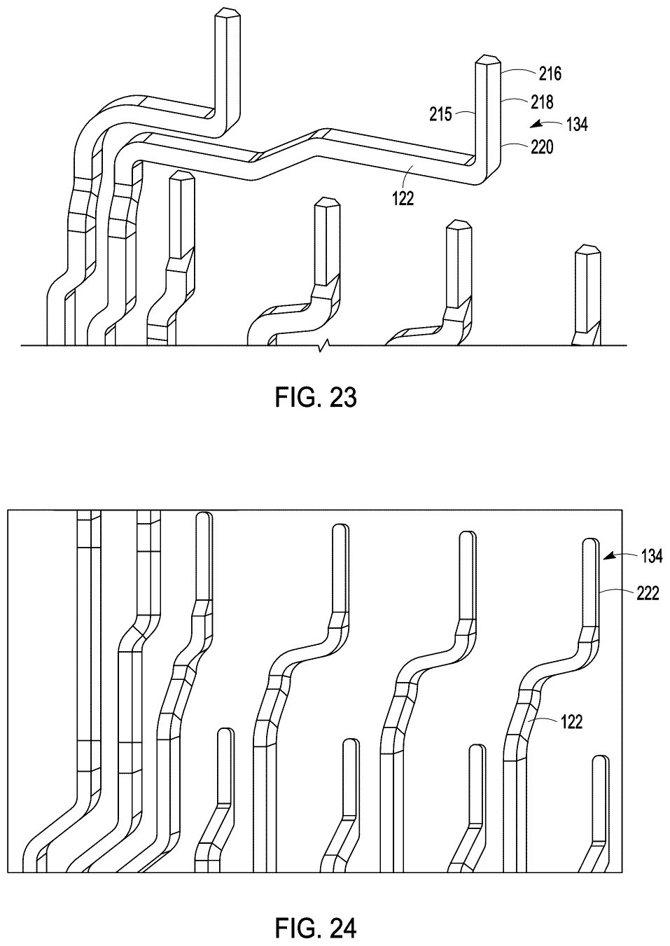

FIG. 23 shows a perspective view of an electrical contact connection section 134, in accordance with one embodiment.

In this embodiment, a connection section 216 can include a chamfered shape extending from a thick side 215 down to a thinned portion 218 and narrowing down to a thin connection portion 220. The connection portion 220 is the very edge, or thinnest portion, of the connection portion 216. In use, the connector 122 will be connected to the electrical contact 119 right on the edge of the chamfered connection section 216 at connection portion 220.

FIG. 24 shows a perspective view of an electrical contact connection section 134, in accordance with one embodiment.

In this example, another example of a flat pad connector 222 is shown, Here, the second end connection sections 134 for connecting to the electrical connection blocks of the header can include a thinned portion defining the flat connection pad 222 that is thinner than a main portion of the connector 122. In this example, the width of the thinned connector pad 222 can be less than a width of the main body of the connector 122.

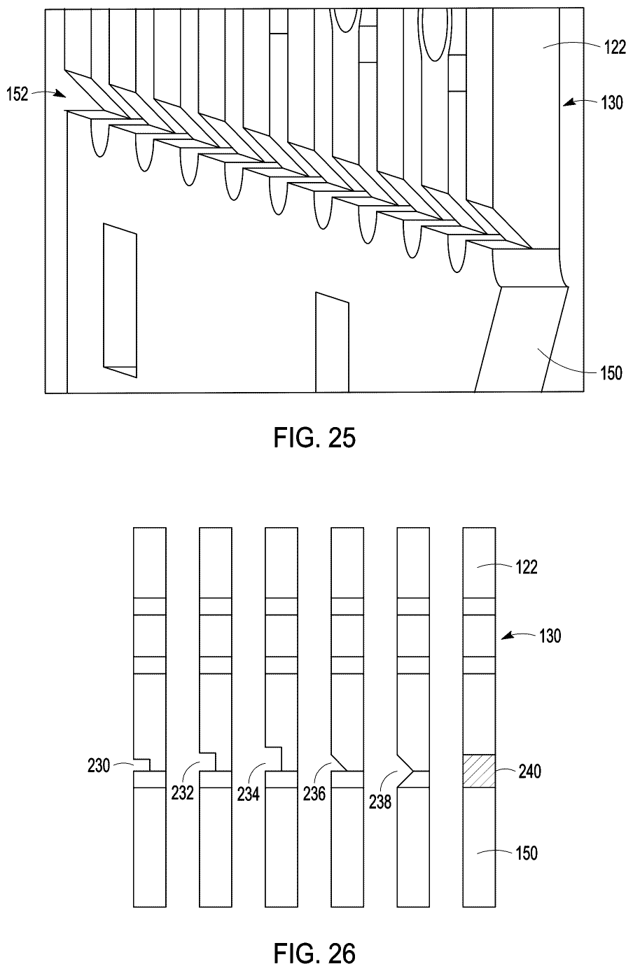

FIGS. 25-29 show further features of the connector assembly 130, described above in FIGS. 4-5.

FIG. 25 shows a perspective view of a connection side of a portion of the connector assembly 130, in accordance with one embodiment.

In this example, the connector assembly 130 includes the break-off features 152 located between the plurality of connectors 122 and the common sacrificial section 150.

FIG. 26 shows a series of side view of a portion of a plurality of connector assemblies 130, in accordance with various embodiments.

The break-off feature 152 can be formed with various different structures during the additive manufacturing process. For example, the break-off feature can include a rectangular break-off feature 230, a larger rectangular break-off feature 232, and an even larger rectangular break-off feature 234. Some embodiments can include a triangular notched break-off feature 236, or a V-shaped triangular break-off feature 238. In one example, the break-off feature can include a weakened section 240 between the connectors 122 and the sacrificial section 150. For example, the break-off feature 240 can be formed of a weaker, less dense material than the sacrificial section 150 and the connectors 122 such that under bending stress the break-off feature will break off from the connectors 122.

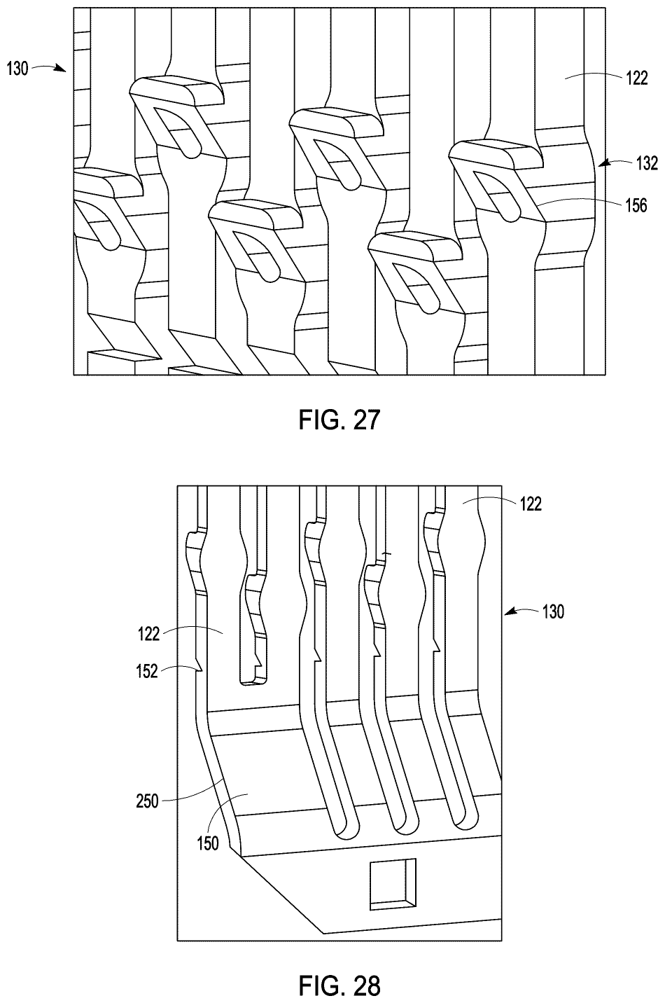

FIG. 27 shows a perspective view of a front side of a lower portion of a connector assembly 130, showing further details of the stand-offs 156, in accordance with one embodiment.

In this example, the stand-offs 156 can be provided to rest against the feedthrough assembly (see FIG. 2). The stand-offs 156 can extend from a back, connection side of the connector assembly 130 proximate the break-off feature 152 and can be located at the connection sections 132 on the first end of the connector assembly 130. The stand-offs 156 rest against the flat ceramic surface of the feedthrough assembly and ensure that the connector sections 132 are at a proper height for optimal connection to the feedthrough pins. Further, after the connection, the stand-offs 156 can provide for added leverage when the sacrificial section 150 is being separated at the break-offs 152.

FIG. 28 shows a perspective view of a lower portion of a connector assembly 130, in accordance with one embodiment.

In this example, at least two of the connectors 122 are attached below the break-off 152 at a double base 250 portion of the sacrificial portion 150 to provide for additional base support for the connectors. This allows for easier leverage when breaking off the sacrificial portion. For example, in this embodiment, the two connectors 122 on the left side of the connector assembly 130 are coupled together all the way up to the break-off feature 152 while the rest of the connectors 122 separate just above a common section of sacrificial portion 150. These two connectors are the longest connecters (see FIG. 2) and are therefore the most fragile. When bending off the sacrificial section 150, the double base 250 allows for the extra leverage so that they break at the break-off feature 152 and not elsewhere along the connector 122.

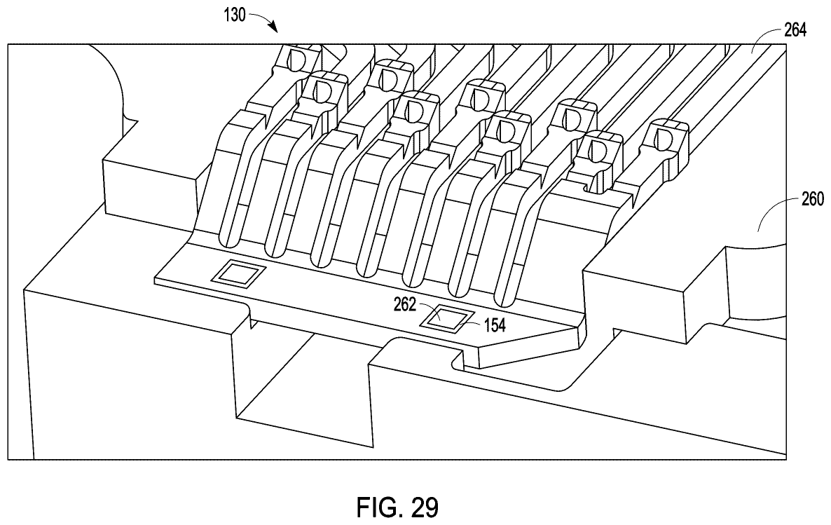

FIG. 29 shows a perspective view of a connector assembly within an assembly fixture 260, in accordance with one embodiment.

In this example, a fixture 260 is shown. In use, the connector assembly 130 fits with grooves 264 in the fixture 260. Moreover the fixture 260 can include posts 262 keyed to receive the keyed holes 154 of the connector assembly 130. During manufacture, the fixture 260 aligns the connector assembly 130 to the implantable device to allow the weld connections to be made.

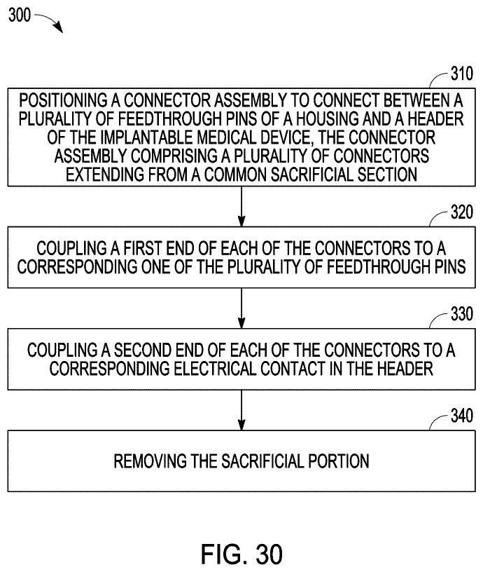

FIG. 30 shows a method (300) for positioning a connector assembly relative to an implantable medical device, in accordance with one embodiment.

In this example, the method (300) can include positioning a connector assembly (310) to connect between a plurality of feedthrough pins of a housing and a header of the implantable medical device, the connector assembly comprising a plurality of connectors extending from a common sacrificial section, the connectors being pre-formed into a 3-dimensional shape such that one or more of the connectors lie in a different plane from a side view than others of the plurality of connectors. The method (300) further includes coupling a feedthrough connection section of each of the connectors to a corresponding one of the plurality of feedthroughs (320), coupling a second end of each of the connectors to a corresponding electrical contact in the header (330), and removing sacrificial portion (340).

As noted, above, the sacrificial portion can include one or more keyed holes to couple to a fixture configured for aligning the connector assembly with the feedthroughs and the electrical contacts.

Also, as noted, the present design can include forming the connector assembly 130 using additive manufacturing such as direct laser melting sintering, selective laser melting, or direct metal laser sintering. The connector assembly 130 can be formed of Niobium or other biocompatible conductor materials such as Ti, Pt, etc. In other uses for outside the body, the connector assembly 130 can be formed of any conductive material.

In summary, the present system can utilize additive manufacturing such as direct laser melting sintering, selective laser melting, or direct metal laser sintering to form a connector assembly. This allows for varied shapes of the connector assembly as a whole in either or both the profile or cross-section to provide for improved manufacture. Moreover, connection sections on each end of the connectors can be designed to provide a more robust weld connection between the connectors and the feedthrough pins and the connector blocks of the header.

### ADDITIONAL NOTES

The above detailed description includes references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents incorporated by reference, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to comply with 37 C.F.R. § 1.72(b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

What is claimed is:

1. An implantable medical device comprising:
a housing including electronic devices within the housing;
a header attached to the housing and including one or more bores; and
a plurality of connectors extending from the housing to the header and coupled to a plurality of electrical contacts within the one or more bores;
wherein at least one of the plurality of connectors includes a first end coupled to a feedthrough pin on the housing and second end coupled to one of the plurality of electrical contacts, wherein the first end includes a contoured shape configured to provide a feedthrough connection section having a non-uniform cross-section.

2. The implantable medical device of claim 1, wherein the contoured shape includes a conical shape having a first wide end on a connection side of the connection section and extending to an open hole on an outer surface of the connection section such that the feedthrough pin is exposed through the open hole.

3. The implantable medical device of claim 1, wherein contoured shape includes a thinned portion of the feedthrough connection section that is thinner in a cross-section than a main portion of the connector, wherein the thinned portion includes a flattened area of the connection section.

4. The implantable medical device of claim 1, wherein the contoured shape includes a pocket pad defining a generally circular recessed portion on a feedthrough pin connection side of the connector to receive the feedthrough pin and leading to a thinned portion located on an outer side of the connector.

5. The implantable medical device of claim 1, wherein the contoured shape includes a pocket pad defining a generally lengthened oval shaped recessed portion on a feedthrough pin connection side of the connector to receive the feedthrough pin and leading to a thinned portion located on an outer side of the connector.

6. The implantable medical device of claim 1, wherein the contoured shape includes a thinned portion of the feedthrough connection section that is thinner in a cross-section than a main portion of the connector, wherein the thinned portion includes flat pads.

7. The implantable medical device of claim 6, wherein the thinned portion includes flat pads that are wider that a main portion of the connector.

8. The implantable medical device of claim 1, wherein the contoured shape includes a thinned portion of the feedthrough connection section that is thinner in a cross-section than a main portion of the connector, wherein the contoured shape of the thinned portion includes swaged contact pads.

9. The implantable medical device of claim 1, wherein the contoured shape includes a thinned portion of the feedthrough connection section that is thinner in a cross-section than a main portion of the connector, wherein the feedthrough connection section includes an inverse pocket structure including the thinned portion with gussets located around a periphery of the thinned portion to stiffen the feedthrough connection section.

10. The implantable medical device of claim 1, wherein the contoured shape includes a hole extending though the feedthrough connection section.

11. The implantable medical device of claim 10, wherein the hole includes a chamfered portion around a periphery of the hole on an outer surface of the feedthrough connection section.

12. An implantable medical device comprising:
a housing including electronic devices within the housing;
a header attached to the housing and including one or more bores; and
a plurality of connectors extending from the housing to the header and coupled to a plurality of electrical contacts within the bore;
wherein at least one of the plurality of connectors includes a first end coupled to a feedthrough pin on the housing and second end coupled to one of the plurality of electrical contacts, wherein the second end includes a contoured shape configured to provide an electrical contact connection section having a non-uniform cross-section.

13. The implantable medical device of claim 12, wherein the contoured shape includes a thinned portion that is thinner than a main portion of the connector, wherein the thinned portion includes a flat surface having a width that is wider than the main portion of the connector.

14. The implantable medical device of claim 12, wherein the contoured shape includes an opening including a slot in the connector to allow for a line-of-site weld connection.

15. The implantable medical device of claim 12, wherein the contoured shape includes a thinned portion that is thinner than a main portion of the connector, wherein the thinned portion includes a chamfered shape narrowing down to a thin connection section on an edge of the connector.

16. A method for positioning a connector assembly relative to an implantable medical device, the method comprising:
positioning a connector assembly to connect between a plurality of feedthrough pins of a housing and a header of the implantable medical device, the connector assembly comprising a plurality of connectors extending from a common sacrificial section, the connectors being pre-formed into a 3-dimensional shape such that one or more of the connectors lie in a different plane from a side view than others of the plurality of connectors;
coupling a first end of each of the connectors to a corresponding one of the plurality of feedthrough pins;
coupling a second end of each of the connectors to a corresponding electrical contact in the header; and
removing the sacrificial section.

17. The method of claim 16, wherein the connector assembly includes a main connector portion which is separated from the sacrificial section by a break-off feature.

18. The method of claim 17, wherein the connector assembly includes a standoff extending from a back surface of the connector proximate the break-off feature.

19. The method of claim 16, wherein sacrificial portion includes one or more keyed holes to couple to a fixture configured for aligning the connector assembly with the feedthrough pins and the electrical contacts.

20. The method of claim 16, including forming the connector assembly using additive manufacturing.
